(19) United States
(12) Patent Application Publication
(10) Pub. No.: US 2008/0269781 A1
(43) Pub. Date: Oct. 30, 2008

(54) Surgical Fastener, Surgical Fastener Kit and Removing Tool

(75) Inventors: Shigeaki Funamura, Fukuroi-shi (JP); Hitoshi Mabuchi, Fukuroi-shi (JP)

Correspondence Address: TYCO HEALTHCARE - EDWARD S. JARMOLOWICZ, 15 HAMPSHIRE STREET, MANSFIELD, MA, 02048, US

(73) Assignee: TYCO HEALTHCARE GROUP LP, Mansfield, MA (US)

(21) Appl. No.: 11/912,033

(22) PCT Filed: Apr. 20, 2006
(86) PCT No.: PCT/EP2006/003649

§ 371 Date: Jan. 14, 2008

(30) Foreign Application Priority Data
Apr. 21, 2005 (JP) 2005-123862

(51) Int. Cl. A61B 17/06; A61B 17/08; A61B 17/32

(52) U.S. Cl. 606/138; 606/215; 606/167

(57) ABSTRACT

The present invention provides an organ fixture having a rod shaped anchoring portion formed by bundling a suturing thread, and a fixing portion made of a suturing thread continuous with the suturing thread of the anchoring portion. By pulling the fixing portion with respect to the anchoring portion with a force larger than a prescribed level, the suturing thread forming the anchoring portion can be loosened. Additionally, the invention provides a type of organ fixture set which pierces and is inserted into the body of the patient while a portion of an anchoring portion and a fixing portion of the organ fixture is accommodated in it, and it can be pulled from the body of the patient while the anchoring portion is left within the stomach wall. Further, the organ fixture set has a unit for removing the organ fixture from the body of the patient.

### FIELD OF THE INVENTION

The present invention pertains to a surgical fastener, or organ fixture, and in particular a surgical fastener suitable for fixing a prescribed organ in the body of a patient on a skin side wall portion, for example for securing a patient's stomach wall to the abdominal wall.

### BACKGROUND OF THE INVENTION

In the prior art, a type of organ fixture set having a fixture inside it for fixing the sutured portion in the body of the patient has been used, especially for fixing skin with internal organs or other organs. For example, for a patient who has lost the ability in take food by mouth on his/her own due to age or disease, a gastrostomy tube is used to feed fluid food, nutrients, and other fluid drinks/foods. However, the gastrostomy tube has to be attached by forming a hole in the abdomen of the patient. In such a case, in order to attach the gastrostomy tube appropriately, it is necessary to make use of an organ fixture set having an organ fixture to fix the abdominal wall and the stomach wall. An example of an organ fixture arrangement is shown in Japanese Kokai Patent Application No. Hei 5[1993]-161655.

The organ fixture set has two piercing needles set parallel to each other with a certain distance between them. When fixing is performed for the abdominal wall and stomach wall, first of all the two piercing needles simultaneously pierce the portion of the patient which is to be sutured. Then, while the suturing thread is made to pass through one piercing needle, an internal needle having a loop member made of a wire connected to its tip portion is made to pass through the other piercing needle; inside the stomach wall, while the suturing thread is held in the loop member, the internal needle is pulled from the piercing needle. Then, while the tip portion of the suturing thread protrudes from the other piercing needle, the two piercing needles are pulled out from the portion to be sutured. Then, the two side portions of the suturing thread protruding out from the body of the patient are tied, and fixing of the abdominal wall and the stomach wall is finished.

However, for the aforementioned conventional organ fixture set, it is necessary to have the internal needle and suturing thread pass through the piercing needles, respectively, while the two piercing needles simultaneously pierce so that the operation becomes complicated, and this is undesirable. Also, if the internal needle is not well inserted into the piercing needle, the protrusion direction and shape of the loop member are in an inappropriate state, and the suturing thread may not be held appropriately. Consequently, much skill is required by the operator. This is also undesirable. Also, because there may be insufficient space inside the portion to be sutured, it is difficult to hold the suturing thread with the loop member, and reliability is decreased. This is undesirable.

The purpose of the present invention is to solve the aforementioned problems of the prior art by providing a type of organ fixture and a type of organ fixture set that can fix the organ in a simple operation with high reliability.

### SUMMARY OF THE INVENTION

The present invention is directed to a surgical fastener comprising an elongate anchoring portion and a fixing portion extending from the anchoring portion wherein the fixing portion is a suturing thread and the anchoring portion is composed of a suture thread bundling, the suture thread of the fixing portion being continuous with the suture thread of the anchoring portion. In the context of the present invention, the term continuous encompasses an arrangement in which a suturing thread of the anchoring portion is joined to a suturing thread of the fixing portion.

Further, the invention provides a sterile surgical kit comprising one or more surgical fasteners and an inserting unit.

Further still, the invention provides a removing unit for removing a surgical fastener inserted in a patient, the removing unit comprising a grooved needle portion.

The invention also provides a method of securing a hollow organ of a patient to a skin side wall portion comprising the steps of: (a) inserting an insertion device carrying a surgical fixture through a patient's skin and into said hollow organ; (b) deploying said surgical fixture; (c) removing said insertion device leaving said surgical fixture in said patient; (d) repeating steps (a)-(c) at least once; and (e) securing together fixing portions of a plurality of surgical fixtures inserted into said patient.

In another embodiment, the invention provides a method of removing a surgical fixture comprising the steps of, (a) inserting a removal device into a patient such that a fixing portion of said surgical fixture enters a grooved portion of said removal device; b) applying tension to said fixing portion such that an anchoring portion of said surgical fixture becomes loosened; c) continuing the application of tension to said fixing portion until said surgical fixture is fully removed from said patient; and d) removing said removal device from said patient.

In yet another embodiment, the invention provides an organ fixture formed by using suturing thread characterized by an anchoring portion and a fixing portion extending from and axial to the anchoring portion through a hole in a patient.

### BRIEF DESCRIPTION OF THE FIGURES

Preferred embodiments of the invention will now be described, by way of example only, with reference to the accompanying drawings in which:

FIG. 1 is a front view illustrating the organ fixture in an embodiment of the present invention.

FIG. 2 is a side view illustrating the organ fixture shown in FIG. 1.

FIG. 3 is a cross-sectional view taken across 3-3 in FIG. 1.

FIG. 4 is a partially cut cross-sectional view illustrating the inserting unit.

FIG. 5 is a partially cut cross-sectional view illustrating the state of attachment of the organ fixture on the inserting unit shown in FIG. 4.

FIG. 6 is a cross-sectional view illustrating the state in which the inserting unit shown in FIG. 5 is pieces the abdomen.

FIG. 7 is a cross-sectional view illustrating the state in which the anchoring portion is pressed by the cylindrical extruder part into within the stomach wall.

FIG. 8 is a cross-sectional view illustrating the state in which the anchoring portion is inserted within the stomach wall.

FIG. 9 is a cross-sectional view illustrating the state of engagement of the anchoring portion on the interior of the stomach wall.

FIG. 10 is a cross-sectional view illustrating the state in which the stomach wall is fixed on the abdominal wall by means of the suturing portion.

FIG. 11 is a plan view illustrating the state in which two suturing portions are formed on the abdomen.

FIG. 12 is a front view illustrating the removing unit.

FIG. 13 is a bottom view of FIG. 12.

FIG. 14 is a cross-sectional view illustrating the state in which the removing unit is inserted into the hole along the fixing portion.

FIG. 15 is a cross-sectional view illustrating the state in which the anchoring portion is loosened.

FIG. 16 is a cross-sectional view illustrating the state in which the organ fixture with the anchoring portion loosened is removed.

FIG. 17 is an oblique view illustrating the inserting unit used in the organ fixture set in another embodiment.

### DETAILED DESCRIPTION OF THE INVENTION

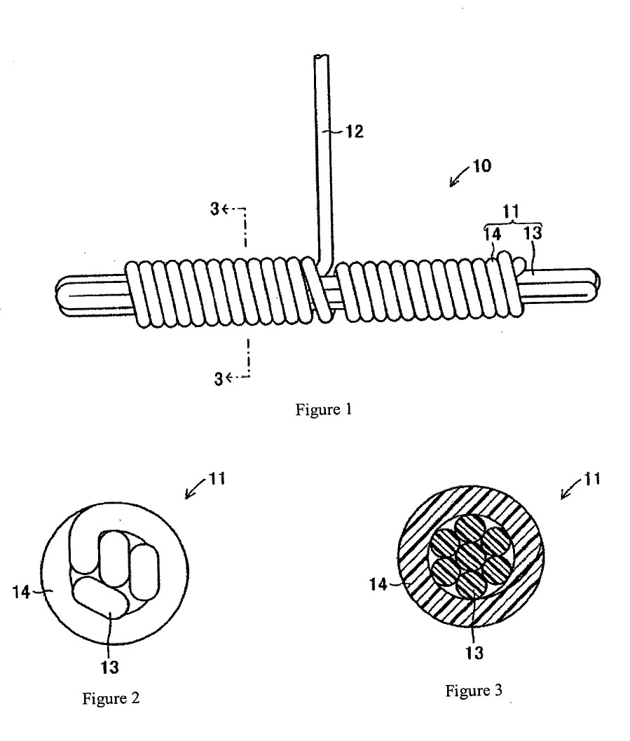

FIG. 1 is a diagram illustrating organ fixture (10) of this embodiment. For example, this organ fixture (10) is for suturing and fixing abdominal wall A and stomach wall B (see FIG. 6 or the like). It is composed of anchoring portion (11) formed in a rod shape and fixing portion (12) that extends perpendicular to anchoring portion (11) from the central portion of the axial direction of anchoring portion (11). For said organ fixture (10), rod-shaped anchoring portion (11) is formed by bundling the suturing thread made of nylon, and the end portion side of the suturing thread is made to extend from the center perpendicular to anchoring portion (11) to form fixing portion (12). In this way, a T-shape is formed.

For anchoring portion (11), one end portion of the suturing thread is folded multiple times in a prescribed length to form core portion (13), and the suturing thread is wound on the periphery of core portion (13) to form winding portion (14). FIG. 2 is a side view of anchoring portion (11). FIG. 3 is a cross-sectional view of anchoring portion (11). Also, fixing portion (12) extends from the center of anchoring portion (11), and the end portion on its side near anchoring portion (11) is connected to core portion (13) while it is fastened with winding portion (14). For organ fixture (10) with said constitution, when fixing portion (12) is pulled with respect to anchoring portion (11) with a force over a prescribed level, core portion (13) is sequentially pulled out from winding portion (14), and the suturing thread that forms anchoring portion (11) is sequentially loosened.

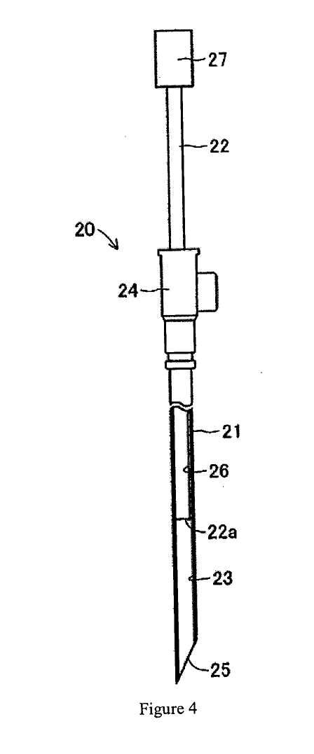

When said organ fixture (10) is attached to the abdomen of the patient, the inserting unit (20) shown in FIG. 4 is used. Said inserting unit (20) is composed of piercing needle (21) for insertion and cylindrical extruder part (22) inserted in said piercing needle (21) for insertion. Said piercing needle (21) for insertion is made of a metal cylindrical body having inserting hole (23) inside it, and it has holding part (24) attached to the base end portion (upper end portion shown in FIG. 4). Said holding part (24) is formed in a cylindrical shape with a larger diameter for the upper side and a smaller diameter for the lower side, and it has a guide hole (not shown in the figure) formed and connected to inserting hole (23).

The diameter of the guide hole is selected to be larger than the diameter of inserting hole (23). As a result, cylindrical extruder part (22) can be inserted easily from the upper end portion of piercing needle (21) for insertion. Also, the diameter of inserting hole (23) is selected so that it allows insertion while anchoring portion (11) of fixing portion (12) of organ fixture (10) are linearly aligned. Also, tip portion (25) of piercing needle (21) for insertion is cut oblique to the axial direction to make it sharp, and at the same time an opening is formed in the lateral direction.

Said cylindrical extruder part (22) is a cylindrical body made of a metal with inserting hole (26) formed in it, and holding part (27) is attached to the base end portion (upper end portion). Inside said holding part (27), a guide hole (not shown in the figure) is formed and connected to inserting hole (26). Also, the outer diameter of cylindrical extruder part (22) is selected to enable insertion into inserting hole (23) of piercing needle (21) for insertion, and the diameter of inserting hole (26) is selected to ensure that fixing portion (12) can pass through its interior, while anchoring portion (11) cannot pass through it.

When organ fixture (10) is attached to inserting unit (20) with the aforementioned constitution, first of all, in inserting hole (23) of piercing needle (21) for insertion while cylindrical extruder part (22) is removed, anchoring portion (11) is inserted from the upper end side of inserting hole (23) while it is set in the longitudinal orientation. Then, the tip portion of fixing portion (12) is inserted from the tip opening of cylindrical extruder part (22), and it protrudes from the upper end portion of the guide hole of holding part (27). Then, tip portion (22a) of cylindrical extruder part (22) in this state is inserted from the upper end portion of the guide hole of holding part (24), and cylindrical extruder part (22) enters piercing needle (21) for insertion.

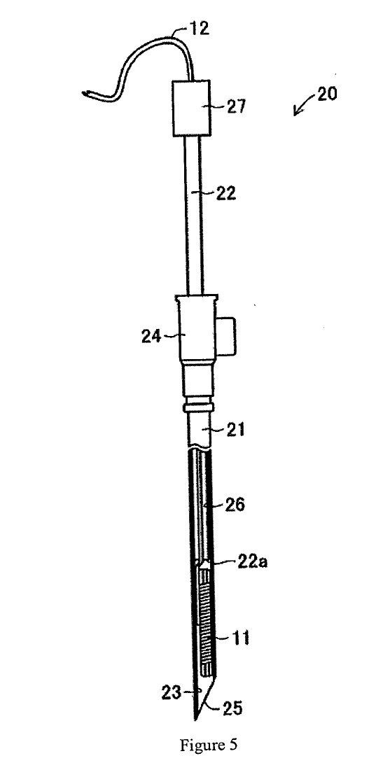

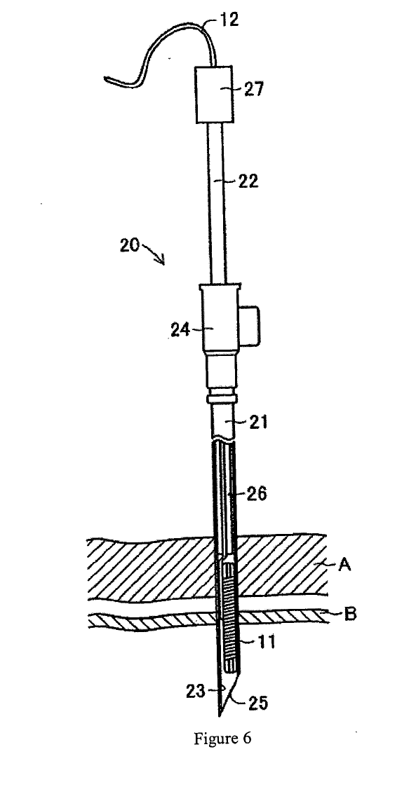

In this case, the end portion side of fixing portion (12) is lightly pulled so that fixing portion (12) does not bend, Then, while anchoring portion (11) is pressed with tip portion (22a) of cylindrical extruder part (22), cylindrical extruder part (22) is driven to move toward the side of tip portion (25) of piercing needle (21), and the tip portion of anchoring portion (11) (the lower end portion when inserted in inserting hole (23)) is positioned near the lower end portion of inserting hole (23), and insertion of cylindrical extruder part (22) stops in the state shown in FIG. 5.

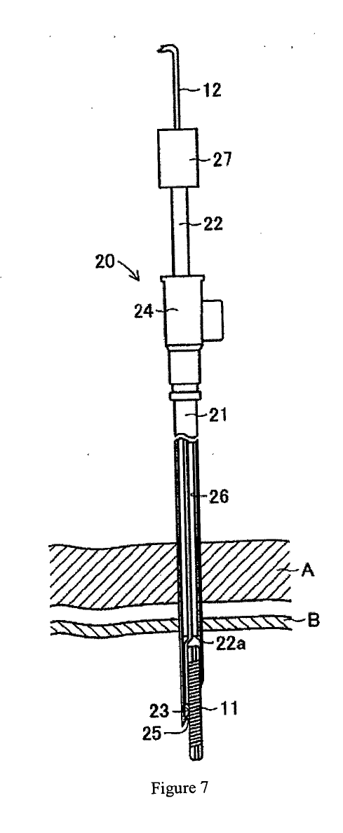

Then, in this way, inserting unit (20) having organ fixture (10) attached to it pierces the surface of the skin of the abdomen of the patient, and as shown in FIG. 6, inserted piercing needle (21) goes through abdominal wall A and stomach wall B. In this case, inserting unit (20) is pressed in until the opening of tip portion (25) of piercing needle (21) is positioned on the inner side of stomach wall B. Then, cylindrical extruder part (22) is pressed into piercing needle (21) for insertion and, as shown in FIG. 7, anchoring portion (11) protrudes from tip portion (25) of piercing needle (21).

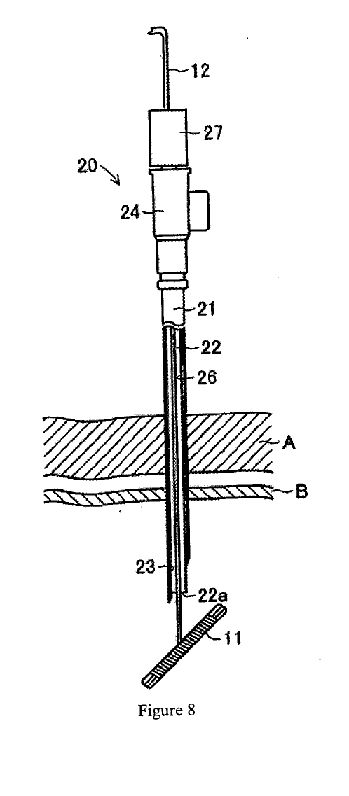

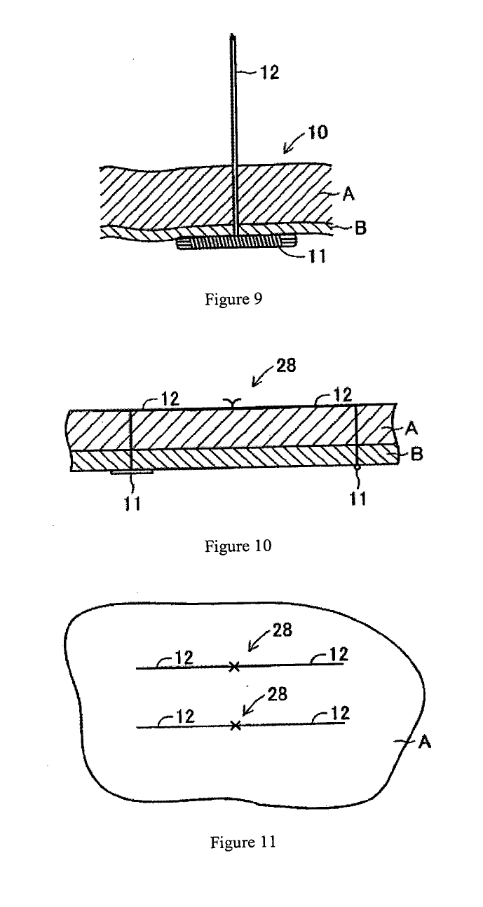

Then, as cylindrical extruder part (22) is further pressed into piercing needle (21) for insertion, as shown in FIG. 8, tip portion (22a) of cylindrical extruder part (22) becomes positioned at tip portion (25) of piercing needle (21). As a result, anchoring portion (11) is pressed to out of piercing needle (21), and it is positioned on the inner side of stomach wall B. In this state, while organ fixture (10) is left in the body of the patient, piercing needle (21) together with cylindrical extruder part (22) is pulled out of the body of the patient. In this case, as shown in FIG. 9, anchoring portion (11) extends in the lateral direction along stomach wall B, and it becomes engaged to stomach wall B.

In this way, said operation is repeated on the portion near organ fixture (10) in the abdomen of the patient having organ fixture (10) attached to it to attach the other organ fixture (10). The end portions of fixing portions (12) of said two organ fixtures (10) out of the body of the patient are tied to each other to form the portion to be sutured (28) shown in FIG. 10. As a result, stomach wall B is fixed on abdominal wall A. Also, as needed, another portion to be sutured (28) may be formed near the portion to be sutured (28), and the state shown in FIG. 11 is formed. In this way, while stomach wall B is fixed on abdominal wall A, the next treatment for attaching the gastrostomy tube or the like can be performed.

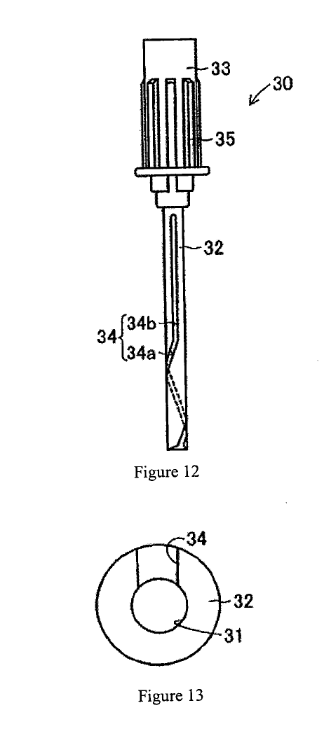

After the end of the prescribed treatment, when organ fixture (10) is removed from the body of the patient, the removing unit (30) shown in FIG. 12 is used. This removing unit (30) is composed of cylindrical portion (32) having a hole (31) (see FIG. 13) formed in it and holding portion (33) set on the base end portion of cylindrical portion (32). In cylindrical portion (32), groove portion (34) is formed from the lower end portion to the upper end portion by piercing between the outer and inner peripheral surfaces. The lower side portion of said groove portion (34) is made of spiral groove (34a) extending in the axial direction while it goes around the circumference, and the upper side portion of groove portion (34) is made of linear groove (34b) that extends in the axial direction. Then, cylindrical holding portion (33) is formed, and embossed portion (35) for inhibiting slipping of the peripheral surface is formed.

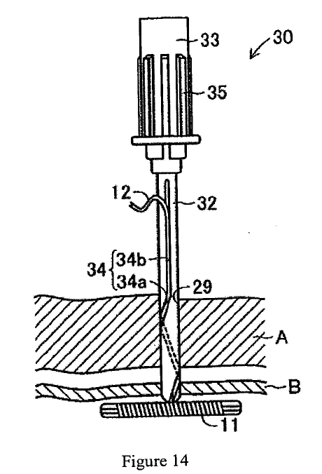

When removing unit (30) with the aforementioned constitution is used to remove organ fixture (10) from the body of the patient, first of all the exposed portion of fixing portion (12) in the portion to be sutured (28) is cut, and the end portion of fixing portion (12) of each organ fixture (10) is released. Then, as shown in FIG. 14, the lower end portion of groove portion (34) is in contact with fixing portion (12), and a portion of fixing portion (12) enters groove portion (34). In this state, fixing portion (12) is wound on the periphery of cylindrical portion (32) of removing unit (30), and while it enters groove portion (34), cylindrical portion (32) is inserted into hole portion (29) formed between abdominal wall A and stomach wall B.

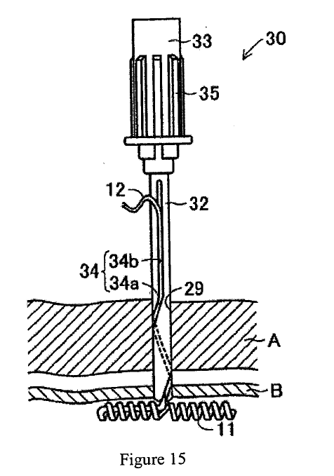

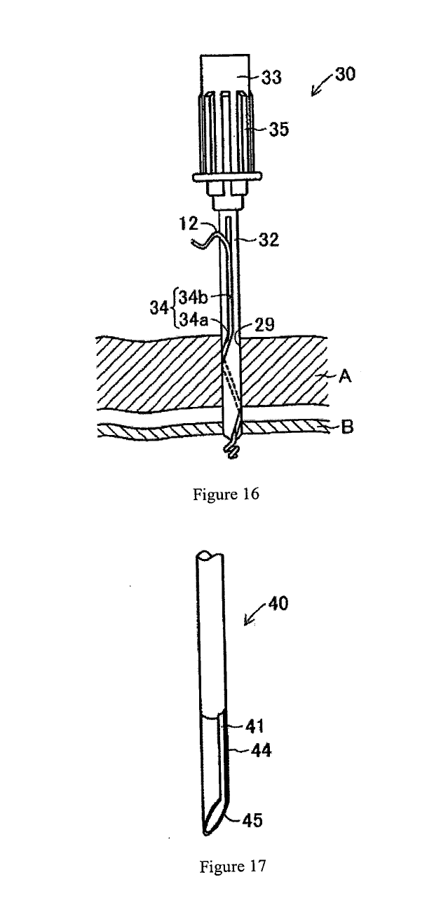

Then, while the tip portion of cylindrical portion (32) is in contact with anchoring portion (11), insertion of cylindrical portion (32) into hole (29) stops, and while removing unit (30) is immobile, fixing portion (12) is pulled. As a result, as shown in FIG. 15, core portion (13) of anchoring portion (11) is pulled out from winding portion (14), and anchoring portion (11) is loosened. Then, by pulling fixing portion (12), as shown in FIG. 16, the loosened anchoring portion (11) is sequentially taken out through the interior of cylindrical portion (32). Then, when the suturing thread that forms organ fixture (10) is entirely taken out, removing unit (30) is pulled from the body of the patient. Similarly, another organ fixture (10) is also removed from the body of the patient, and the removal operation of organ fixture (10) comes to an end. In this embodiment, the organ fixture set of the present invention is composed of organ fixture (10), inserting unit (20) and removing unit (30).

As explained above, said organ fixture (10) is composed of anchoring portion (11) in a rod shape by bundling a suturing thread, and fixing portion (12). Consequently, when anchoring portion (11) is positioned inside stomach wall B, anchoring portion (11) goes along the inner wall of stomach wall B, anchoring portion (11) and fixing portion (12) form a T shape, and the end side portion of fixing portion (12) protrudes from the body of the patient. Consequently, by attaching two organ fixtures (10) at a prescribed position of the abdomen, and tying fixing portions (12) protruding out of the body of the patient, the portion to be sutured (28) is formed, and stomach wall B can be fixed on abdominal wall A. Also, when organ fixture (10) is to be removed, by simply loosening anchoring portion (11) and pulling fixing portion (12), organ fixture (10) can be removed from hole portion (29).

For the organ fixture set of the present invention, when organ fixture (10) is attached to the abdomen of the patient, while a portion of anchoring portion (11) and fixing portion (12) are inserted into piercing needle (21) of inserting unit (20), piercing needle (21) pierces the abdomen of the patient, so that anchoring portion (11) is inserted into stomach wall B. Then, while the portion of the side of tip portion (25) of piercing needle (21) is positioned within the stomach wall B, after anchoring portion (11) is pressed out of piercing needle (21) with cylindrical extruder part (22), inserting unit (20) is pulled from the body of the patient, and it is possible to attach organ fixture (10). In this way, with a simple operation, organ fixture (10) can be attached to the body of the patient.

Then, when the organ fixture set of the present invention is used to remove organ fixture (10) from the abdomen of the patient, by using removing unit (30), a simple operation can remove organ fixture (10). That is, while the exposed portion of fixing portion (12) is wound on the peripheral surface of cylindrical portion (32) of removing unit (30) and is put in groove portion (34), cylindrical portion (32) is inserted into hole portion (29), and while the tip portion of cylindrical portion (32) is in contact with anchoring portion (11), by pulling fixing portion (12), it is possible to take out organ fixture (10) easily.

Also, FIG. 17 is a diagram illustrating inserting unit (40) used in the organ fixture set in another embodiment of the present invention. This inserting unit (40) is made of a cylindrical body having hole (41) formed in it, and tip portion (45) is cut oblique to the axial direction to form a sharp tip. Also, groove portion (44) is formed extending straight from the upper end portion of the opening of tip portion (45) toward the upper end of inserting unit (40). When this inserting unit (40) is used to attach organ fixture (10) to the body of the patient, while fixing portion (12) is on the outside, anchoring portion (11) is put in groove portion (44) from tip portion (45) of inserting unit (40). Then, after inserting unit (40) pierces the body of the patient, anchoring portion (11) is pressed into stomach wall B, and inserting unit (40) is pulled out from the body of the patient so that organ fixture (10) is attached. As a result, attachment of organ fixture (10) can be performed easily.

In yet another embodiment, the suturing thread that forms anchoring portion (11) of organ fixture (10) is fixed by an adhesive made of a bioabsorbable material that can disappear over a prescribed time. In this case, at a prescribed time after attachment of organ fixture (10), because anchoring portion (11) is loosened, organ fixture (10) can be easily removed from the body by simply pulling fixing portion (12), without using removing units (30), (40).

The organ fixture and organ fixture set of the present invention are not limited to the aforementioned embodiments. One may make appropriate changes for its embodiment. For example, in said embodiments, the suturing thread for forming organ fixture (10) is made of nylon. However, the material of the suturing thread is not limited to nylon. One may also make use of polyester, silk, polyolefin, as well as bioabsorbable materials such as a Lactomer or a Glycomer. If desired, the fixing portion and the securing portion may be made of differing suture threads. Also, the scheme for bundling anchoring portion (11) is not limited to the aforementioned embodiment. One may also adopt any scheme that can form anchoring portion (11) in a rod shape.

In this case, the rod shape may be formed using an adhesive made of a bioabsorbable material or the like. In addition, appropriate changes may be made in the shape and material and other structural features of the inserting and removing units, etc. For example, examples of the materials that can be used in forming the inserting unit include stainless steel, Nitinol, titanium, and other metallic materials, as well as polycarbonate, nylon, fluororesins and other resin materials. In addition, the organ fixture and organ fixture set of the present invention are not limited to suturing of abdominal wall A and stomach wall B. It may also be adopted to suturing the kidneys, bladder, and other organs in the human body.

Such a type of organ fixture may be characterized by the following facts: the organ fixture is for fixing a prescribed organ in the body of the patient on the inner wall side of said organ; and it is composed of an anchoring portion that is formed in a rod shape by bundling the suturing thread and is set on the inner wall side of said organ, and a fixing portion, which is composed of a suturing thread continuous with the suturing thread that forms said anchoring portion, and which is set extending from near the center in the axial direction of said anchoring portion through a hole portion formed between said organ and said skin side wall portion to outside the body of the patient.

In a preferred arrangement, the organ fixture of the present invention with the aforementioned constitution is composed of a bundle-shaped anchoring portion set on the inner wall side of the organ and a fixing portion that is set from nearly the center of the anchoring portion through a hole portion formed on the patient and extends to outside the body of the patient. Here, said nearly the center of the anchoring portion in the axial direction is nearly the center of the longitudinal direction, and it is the position where the anchoring portion is opened with respect to the fixing portion when the anchoring portion is positioned inside the organ. Consequently, when the anchoring portion goes through the hole portion and is positioned inside the organ, the anchoring portion goes along the inner wall of the organ, the anchoring portion and fixing portion form a T-shape, so that the organ and the skin side wall portion are fixed. Consequently, the structure of the organ fixture set becomes simpler, and the operation for attaching the organ fixture set becomes much simpler.

Also, since the anchoring portion is formed in a rod shape as the suturing thread is bundled, when the organ fixture set is removed, it can be removed easily from the hole portion by loosening the anchoring portion and pulling the fixing portion. In this case, for example, a bioabsorptive material may be used as the adhesive to form the rod-shaped anchoring portion. As a result, since the anchoring portion is set in the prescribed organ in the body, after a prescribed time the anchoring portion becomes loosened. Consequently, after the prescribed treatment, such as attachment of the gastrostomy tube or other medical device is finished, one may simply pull the fixing portion while the organ fixture is removed.

Also, the constitution is such that the suturing threads that form said anchoring portion can be loosened by pulling said fixing portion with respect to said anchoring portion with a force larger than a prescribed level. In this case, the force larger than a prescribed level is the force larger than the force of the anchoring portion in fixing the organ. One may also adopt a scheme in which the force is larger than the force of the anchoring portion in fixing the organ only during a prescribed time after the anchoring portion has fixed the organ, and after a prescribed time the force is made smaller than the force for fixing the organ, or a near-zero force. As a result, after the gastrostomy tube or other medical device is attached, the removal operation when the organ fixture is removed becomes easier and has high reliability.

As another characteristic feature of the organ fixture of the present invention, said anchoring portion is composed of a core portion formed by folding said suturing thread multiple times to form a bundle with a prescribed length, and a winding portion formed by winding the suturing thread on the periphery of said core portion. As a result, manufacturing of the organ fixture becomes easier, and at the same time the operation for loosening the anchoring portion also becomes easier.

As another characteristic feature of the organ fixture set of the present invention, the organ fixture set has a type of inserting unit for attaching said organ fixture to said organ; and said inserting unit is made of a member that can be inserted into the body of the patient while the anchoring portion of said organ fixture is accommodated in it and can be pulled out from said patient while said anchoring portion is left inside said organ. In this case, the inserting unit may have a constitution made of a needle that can pierce and be insert into the body of the patient, or a constitution made of a cylindrical member without a sharp tip that can be inserted into a hole formed on the body of the patient beforehand. As a result, it is possible to facilitate the operation in attaching the organ fixture into the organ.

As yet another characteristic feature of the organ fixture set of the present invention, the organ fixture set has a type of removing unit for removing said organ fixture from said organ, and said removing unit is made of a cylindrical member which has at least a portion of said organ fixing portion accommodated in it and can be inserted into said hole, and which can be pulled out from said hole after said fixing portion is pulled to loosen said anchoring portion while its tip portion is in contact with said anchoring portion. As a result, the operation for removing the organ fixture becomes easier.

As yet another characteristic feature of the organ fixture set of the present invention, said removing unit is composed of a cylindrical member and a holding part formed on the base end portion of the cylindrical member, and a spiral groove portion extending from the tip portion of said cylindrical member to the base end portion in the axial direction while going around the circumference is formed by piercing between the outer and inner peripheral surfaces of said cylindrical member. As a result, it is possible to insert the removing unit into the body of the patient while rotating it to have the fixing portion enter the groove, and by pressing the tip portion of the removing unit on the boundary portion between the anchoring portion and the fixing portion, it is possible to remove the anchoring portion from the body of the patient while it is effectively loosened.

Other variations and modifications will be recognized by those of ordinary skill in the art as being within the scope of the present invention.

1. A surgical fastener comprising an elongate anchoring portion (11) and a fixing portion (12) extending from the anchoring portion wherein the fixing portion is a suturing thread and the anchoring portion (11) is composed of a suture thread bundling (13, 14), the suture thread of the fixing portion (12) being continuous with the suture thread of the anchoring portion (11).

2. The fastener according to claim 1 wherein said anchoring portion (11) can be loosened by pulling said fixing portion (12) with respect to said anchoring portion with a force larger than a prescribed level.

3. The fastener according to claim 1 or claim 2 wherein said anchoring portion (11) comprises a core (13) of folded suturing thread and a winding portion (14) of suturing thread wrapped around said core portion (13).

4. The fastener according to claim 1 wherein the anchoring portion (11) includes an adhesive made of a bioabsorbable material.

5. The fastener according to claim 4 wherein said anchoring portion (11) comprises a core (13) of folded suturing thread and a winding portion (14) of suturing thread wrapped around said core portion (13).

6. The fastener according to any preceding claim wherein the anchoring portion (11) is substantially rod-shaped and the fixing portion (12) extends substantially perpendicularly from a central region of the anchoring portion (11).

7. The fastener according to any preceding claim wherein the fixing portion (12) of the fastener is secured to a fixing portion (12) of another such fastener.

8. The fastener according to any preceding claim wherein the suturing thread of the anchoring portion (11) is made of a different material to the suturing thread of the fixing portion (12).

9. The fastener according to claim 8 wherein the suturing thread of the anchoring portion (11) is made of a bioabsorbable material.

10. A sterile surgical kit comprising one or more surgical fasteners according to any one of claims 1-9 and an inserting unit (20, 40).

11. The kit according to claim 10 wherein said inserting unit (20) comprises a hollow piercing needle (21) and an extruder (22) within said needle.

12. The kit according to claim 10 wherein said inserting unit (40) comprises a needle element having a hollow tip portion (45) and wherein the hollow tip portion has a side wall incorporating a slot.

13. A removing unit (30) for removing a surgical fastener according to any one of claims 1-9 inserted in a patient, the removing unit (30) comprising a grooved needle portion (32).

14. The removing unit according to claim 13 wherein the grooved needle portion (32) comprises a distal portion and a proximal portion the distal portion having on an outer surface a substantially spiral groove (34a) and the proximal portion having on an outer surface a substantially linear groove (34b) extending in an axial direction of the needle portion (32).

15. A method of securing a hollow organ of a patient to a skin side wall portion comprising the steps of:
(a) inserting an insertion device carrying a surgical fixture according to any one of claims 1-7 through a patient's skin and into said hollow organ,
(b) deploying said surgical fixture,
(c) removing said insertion device leaving said surgical fixture in said patient,
(d) repeating steps (a)-(c) at least once, and
(e) securing together fixing portions of a plurality of surgical fixtures inserted into said patient.

16. The method according to claim 15 wherein the method of securing is a method for securing a patient's stomach wall to the patient's abdominal wall.

17. The method according to claim 14 wherein following the securing of the patient's stomach wall to the patient's abdominal wall, a gastrostomy tube is inserted in to the patient's stomach.

18. A method of removing a surgical fixture according to any one of claims 1-9 comprising the steps of:
(a) inserting a removal device into a patient such that a fixing portion of said surgical fixture enters a grooved portion of said removal device,
(b) applying tension to said fixing portion such that an anchoring portion of said surgical fixture becomes loosened, and
(c) continuing the application of tension to said fixing portion until said surgical fixture is fully removed from said patient, and
(d) removing said removal device from said patient.

19. An organ fixture (10) formed by using suturing thread characterized by an anchoring portion (11) and a fixing portion (12) extending from and axial to the anchoring portion (11) through a hole (29) in a patient.
